(19) United States
(12) Patent Application Publication
(10) Pub. No.: US 2006/0205984 A1
(43) Pub. Date: Sep. 14, 2006

(54) Continuous method for producing methyl vinyl ether

(75) Inventors: Katrin Klass, Mannheim (DE); Heike Becker, Mannheim (DE); Regina Vogelsang, Ludwigshafen (DE); Alexander Hauk, Ludwigshafen (DE); Markus Siegert, Heidelberg (DE); Jochem Henkelmann, Mannheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/554,607

(22) PCT Filed: Apr. 16, 2004
(86) PCT No.: PCT/EP04/04038

§ 371 Date: Oct. 28, 2005

(30) Foreign Application Priority Data
Apr. 30, 2003 (DE) 103 19 452.5

(51) Int. Cl. C07C 41/08

(52) U.S. Cl. 568/688

(57) ABSTRACT

A process for continuously preparing methyl vinyl ether by reacting methanol with ethyne in the liquid phase in the presence of a basic alkali metal or alkaline earth metal compound at a temperature of from 40 to 300° C. and a pressure of from 0.1 to 5 MPa abs, by carrying out the reaction in the absence of a continuous gas phase and at a methyl vinyl ether concentration in the entire liquid phase of ≦30% by weight.

The present invention relates to a process for continuously preparing methyl vinyl ether by reacting methanol with ethyne in the liquid phase in the presence of a basic alkali metal or alkaline earth metal compound at a temperature of from 40 to 300° C. and a pressure of from 0.1 to 5 MPa abs.

Vinyl ethers constitute an important class of compounds having a broad field of use. For instance, they find use, inter alia, as monomer building blocks in polymers and copolymers, in coatings, adhesives, printing inks and in radiation-curable coating materials. Further fields of application are the preparation of intermediates, fragrances and flavors, and also pharmaceutical products.

Vinyl ethers are generally prepared industrially by reacting the appropriate alcohols with ethyne in the presence of basic catalysts (see Ullmann's Encyclopedia of Industrial Chemistry, 6^{th} edition, 2000 Electronic Release, Chapter “VINYL ETHERS—Production” and W. Reppe et al., Justus Liebigs Ann. Chem., 601 (1956), pages 135 to 138). The vinylation can be carried out either in the liquid phase or in the gas phase. In the vinylation in the gas phase, basic heterogeneous catalysts, for example KOH on activated carbon or MgO or CaO, are used. In the liquid phase, the strongly exothermic reaction is generally carried out in the presence of alkali metal hydroxide or alkali metal alkoxide catalysts.

DE-A 100 17 222 describes a process for preparing N-alkenyl ethers by reacting an alcohol with an alkyne in the presence of a basic alkali metal compound and of a mono- or diether of 1,4-butanediol as a cocatalyst. The vinylation is effected batchwise, semibatchwise or continuously, at a temperature of from 100 to 200° C., a partial ethyne pressure of less than 5 MPa and a reaction time of several hours. The examples cited relate to the semibatchwise preparation of tert-butyl vinyl ether and phenyl vinyl ether in an autoclave containing the liquid reaction mixture and a gas phase above it. A disadvantage of the process described is the formation of a gas phase which, depending on the reaction control, may contain a correspondingly high concentration of unconverted ethyne and might thus lead, as a consequence of the tendency of ethyne to decompose, to a safety problem.

SU 481 589 describes a process for preparing N—C₁- to C₅-alkyl vinyl ethers by reacting the corresponding alcohol with ethyne in the presence of a basic alkali metal compound and the high-boiling by-product of the vinylation reaction as a solvent at a temperature of from 100 to 154° C. and a pressure of from 0.7 to 1.3 atmospheres (from about 0.07 to 0.13 MPa abs). A disadvantage of the process described is the use of a solvent which constitutes a further component in the reaction mixture and whose volume requirement leads to a reduction in the space-time yield. In addition, the amount required of the solvent to be used is only obtainable by a complicated vinylation which lasts from 6 to 12 days. Also disadvantageous is the formation of a gas phase which, depending on the reaction control, may contain a correspondingly high concentration of unconverted ethyne and, as a consequence of the tendency of ethyne to decompose, might lead to a safety problem.

DD-A 298 775 and DD-A 298 776 teach processes for preparing N-alkyl vinyl ethers by reacting an alcohol with ethyne in the presence of basic alkali metal compounds, of a crown ether, or a polyethylene glycol, as a cocatalyst, and of a solvent, at a temperature of from 20 to 200° C. and a pressure of from 0.05 to 0.3 MPa abs, at a molar ethyne/alcohol ratio of from 1 to 20. According to the teaching of the documents mentioned, the reaction is to be conducted while maintaining a low alcohol concentration in the reaction mixture, and relatively large amounts of alcohol in the reaction mixture are to be avoided. When preparing low-boiling vinyl ethers, for example methyl vinyl ether, it is appropriate to continuously distill these from the reaction mixture.

U.S. Pat. No. 3,370,095 discloses a process for continuous or batchwise preparation of low molecular weight N-alkyl vinyl ethers by reacting a low molecular weight alkanol with ethyne in the presence or a basic alkali metal compound and of a C₁₀- to C₂₂-alkanol or its vinyl ether as a solvent at a temperature of from 160 to 200° C. and atmospheric pressure, at a molar ratio of the low molecular weight alkanol to ethyne of from 1 to 2. Under the elevated temperature mentioned and the relatively low pressure, the low molecular weight N-alkyl vinyl ether formed evaporates continuously out of the liquid phase together with the excess low molecular weight alkanol and the unconverted ethyne, and is discharged out of the reaction apparatus.

A disadvantage of the process described in DD-A 298 775, DD-A 298 776 and U.S. Pat. No. 3,370,095 is the use of a solvent which constitutes a further component in the reaction mixture and whose volume requirement leads to a reduction in the space-time yield. In addition, as a consequence of the continuous distillation of the low-boiling alkyl vinyl ether out of the reaction mixture, only a relatively small reaction pressure is possible, which leads to low solubility of the ethyne in the liquid phase and thus to a low reaction rate. In addition, the amount of ethyne added has to be kept low, since an ethyne-containing gas phase would otherwise be formed which, as a consequence of the tendency of ethyne to decompose, might lead to a safety problem. Furthermore, the process described in U.S. Pat. No. 3,370,095 entails an increased requirement for ethyne, since the C₁₀- to C₂₂-alkanol used as a solvent is likewise vinylated in the course of the reaction.

EP-A 0 733 401 teaches a process for batchwise reaction of ethyne with an alcohol to give the corresponding vinyl ether in the presence of basic alkali metal compounds in the liquid phase at a temperature of from 0 to 300° C. and a pressure of from 0.2 to 3 MPa abs, in which acetylene is introduced into the liquid phase in the absence of a continuous gas phase under isobaric conditions to a degree of saturation of from 5 to 100%.

A disadvantage of the abovementioned semibatchwise or batchwise processes are the time-consuming and labor- and energy-intensive operation steps of charging, heating, compressing, cooling, decompressing and emptying the reaction apparatus, in particular when preparing amounts which require a plurality of reaction batches. Furthermore, the semibatchwise syntheses run through a large concentration range with regard to the reactant and product concentrations, which leads to a conversion-dependent reaction rate and can support the formation of undesired by-products. The disadvantages mentioned result in a relatively low selectivity and, taking into account the setup times required, also a distinctly lower space-time yield than would be expected on the basis of the chemical reaction rate.

DE ref. no. 101 59 673.1 teaches a continuous process for preparing vinyl ethers by reacting the appropriate alcohol with ethyne in the liquid phase in the presence of a basic alkali metal or alkaline earth metal compound at from 40 to 300° C. and from 0.11 to 5 MPa abs, in which an alcohol conversion of ≧90% is established and operation is effected in the absence of a continuous gas phase.

The invention is based on the recognition that the abovementioned processes, as a consequence of the very low boiling point of methyl vinyl ether of about 6° C., do not present any satisfactory solution for a process which is unproblematic from a safety point of view and at the same time economically viable.

It is an object of the present invention to find a process for preparing methyl vinyl ether by vinylating methanol which does not have the abovementioned disadvantages, is simple to carry out, is unproblematic from a safety point of view, especially with regard to the handling of gaseous ethyne, has a high selectivity for methyl vinyl ether and enables an overall high yield and high space-time yield of methyl vinyl ether.

We have found that this object is achieved by a process for continuously preparing methyl vinyl ether by reacting methanol with ethyne in the liquid phase in the presence of a basic alkali metal or alkaline earth metal compound at a temperature of from 40 to 300° C. and a pressure of from 0.1 to 5 MPa abs, which comprises carrying out the reaction in the absence of a continuous gas phase and at a methyl vinyl ether concentration in the entire liquid phase of ≦30% by weight.

In this context, continuous gas phase refers to gas spaces within the reaction chamber whose size goes beyond individual discrete bubbles. To avoid a continuous gas phase, it is essential to carry out the reaction at a methyl vinyl ether concentration in the entire liquid phase of ≦30% by weight. The relatively low concentration of the volatile methyl vinyl ether mentioned distinctly reduces the vapor pressure of the mixture, so that this decisively counteracts the formation of a gas phase.

Preference is given to carrying out the reaction at a methyl vinyl ether concentration in the entire liquid phase of ≦25% by weight, more preferably ≦20% by weight, even more preferably ≦15% by weight and in particular ≦10% by weight. The lower limit of the concentration of methyl vinyl ether in the entire liquid phase is generally ≧1% by weight, preferably ≧2% by weight and more preferably ≧5% by weight. The proportion missing to 100% by weight in the liquid phase comprises excess methanol, the basic alkali metal or alkaline earth metal compound, dissolved ethyne and any by-products formed, and also any additional solvent added.

Further measures which, in addition to the feature which is essential to the invention of the methyl vinyl ether concentration in the entire liquid phase, supplementarily counteract the formation of a continuous gas phase include precise control of the ethyne concentration in the entire liquid phase within the region of ethyne gas solubility in the liquid phase, intensive mixing of the reaction mixture and a withdrawal means in the top region of the apparatus and lines.

In general, the ethyne is added in an amount of ≦100%, preferably ≦95%, more preferably ≦90% and even more preferably ≦80%, of the maximum gas solubility in the liquid phase. The lower limit of the ethyne concentration in the liquid phase is uncritical in connection with avoidance of a continuous gas phase.

Useful reactors in the process according to the invention are in principle the apparatus described in the technical literature for gas-liquid reactions. In order to supplementarily counteract the formation of a continuous gas phase, it is advantageous to bring about intensive mixing of the reaction mixture. This may be achieved, for example, by efficiently introducing the ethyne into the liquid phase and/or by further measures for mixing the reaction mixture, for instance by intensive stirring or generating intensive flow currents. Intensive mixing of the reaction mixture additionally contributes to an increase in the space-time yield.

The reaction in the process according to the invention can be carried out in a single reactor or in a plurality of reactors connected in series, for example a reactor cascade. Useful reactors include stirred tanks, stirred tank cascades, flow tubes (preferably with internals), bubble columns and loop reactors. In order to achieve efficient introduction, the ethyne, when stirred tanks are used, is preferably introduced via the stirrer, and, when flow tubes, bubble columns and loop reactors are used, preferably via nozzles.

In the process according to the invention, particular preference is given to using a jet loop reactor. The internal insert tube and the feed of the reactants and optionally an external cycle stream via what is known as a jet nozzle generates an internal cycle stream which brings about particularly intensive mixing. In order to ensure the introduction of sufficiently large impulse through the jet nozzle, preference is given to operating the jet loop reactor with an external circuit. This involves continuously withdrawing reaction mixture and recycling it via a pump and optionally a heat exchanger to the jet nozzle. The jet nozzle may be mounted at the bottom or at the top of the reactor, and in the latter case preference is given to a concentric insert tube being disposed in the center of the reactor along the flow direction of the liquid stream introduced. The arrangement of the jet nozzle at the highest point in the reactor has the advantage that when the liquid circulation comes to a stop, no liquid can climb into the gas-conveying part of the jet nozzle. Correspondingly, when a jet nozzle at the top end is used, the withdrawal point of the cycle stream is at the bottom of the reactor. The use of a self-aspirating jet nozzle constitutes an additional, integrated safety feature, since in the event of failure of the circulation pump and thus of the liquid driving jet, no more gas can be aspirated.

In order to reliably prevent a theoretically possible accumulation of gas bubbles at the top of the reactor, the reaction mixture to be withdrawn for the subsequent workup and isolation of the methyl vinyl ether is preferably withdrawn at the highest point of the reactor.

In order to react the dissolved remaining ethyne with the methanol, preference is given to a process in which the reaction is carried out in a main reactor while feeding the starting compounds, methanol and ethyne, and one or more postreactors without further feed of the starting compounds. Useful postreactors include, for example, flow tubes or delay time vessels. In order to prevent backmixing, preference is given to using postreactors in a battery. In general, the postreactors have a smaller volume than the main reactor. To complete the reaction in the postreactor or in the postreactors, a delay time of from 5 minutes to 1 hour is generally sufficient. An ethyne conversion of up to 100% is thus possible.

The reaction in the process according to the invention is carried out at a temperature of from 40 to 300° C., preferably from 60 and 230° C., more preferably from 70 to 200° C. and even more preferably from 80 to 150° C. It is carried out at a pressure of from 0.1 to 5 MPa abs, preferably from 0.15 to 3 MPa abs and more preferably from 0.5 to 3 MPa abs.

Methanol and ethyne are generally fed in accordance with their consumption. The amounts fed therefore correspond approximately to the stoichiometric amounts required, corrected for possible influences such as by discharge of unconverted reactants or by by-product formation.

In the process according to the invention, the basic alkali metal or alkaline earth metal compound is generally used in an amount of from 0.05 to 15% by weight, preferably from 0.1 to 10% by weight and more preferably from 0.1 to 5% by weight, based on the reaction mixture.

The basic alkali metal and alkaline earth metal compounds used in the process according to the invention are generally the oxides, hydroxides and alkoxides, for example the C₁- to C₄-alkoxides such as methoxide, ethoxide, 1-propoxide, 2-propoxide, 1-butoxide, 2-butoxide, 2-methyl-1-propoxide and 2-methyl-2-propoxide, of lithium, sodium, potassium, cesium, magnesium and calcium. Preferred cations are sodium and potassium, in particular potassium. Preferred anions are hydroxides and alkoxides. The alkali metal and alkaline earth metal alkoxides are obtainable, for example, by reacting the alkali metals or alkaline earth metals, their oxides or hydroxides with the appropriate alcohol while removing the by-products formed, hydrogen or water. When oxides and hydroxides are used, they are generally heated together the corresponding alcohol while evaporating the water formed. It is also possible to use mixtures of different alkali metal and alkaline earth metal compounds.

When the oxides or hydroxides are used, they are in chemical equilibrium with the methanol to form water and methoxide. Based on the amount of oxide or hydroxide used, this leads to a relatively small proportion of chemically active methoxide, which requires the use of a distinctly larger amount of oxide or hydroxide for compensation.

In this context, it has also been found that the use of alkoxides results in the occurrence of less by-product formation and a continuous gas phase only occurring at even higher methyl vinyl ether concentrations. The former leads to an advantage in subsequent workup, the latter to a higher safety reserve.

Particular preference is therefore given to using alkoxides, very particular preference to using methoxide and in particular potassium methoxide.

In the process according to the invention, it is possible and in some cases advantageous to use cocatalysts. They may in some cases bring about a reduction in the by-product formation. The use of cocatalysts is common knowledge and is described, for example, in DE-A 3 215 093, U.S. Pat. No. 5,665,889, DE-A 100 17 222, WO 01/46139 and WO 01/46141, which are explicitly incorporated herein by way of reference. Preferred examples of possible cocatalysts include polyoxyalkylene compounds (for example polyoxyethylene or polyoxypropylene) or divinyl compounds of diols (for example 1,2-divinyloxyethane or 1,4-divinyloxybutane). They are generally used in an amount of from 2 to 30% by weight. Depending on the type and amount of the cocatalyst used, it may in some cases also be regarded as a solvent.

In the process according to the invention, it is in principle also possible to use solvents. Suitable solvents have the features that both methyl vinyl ether and methanol, and the basic alkali metal and alkaline earth metal compounds dissolve in them, they do not react chemically with these compounds, i.e. in particular have no acidic centers which would scavenge the basic groups, and they can be removed from the methyl vinyl ether formed without great cost and inconvenience, preferably by distillation. Examples of suitable solvents include the dipolar aprotic solvents N-methylpyrrolidone, tetrahydrofuran and the dialkyl ethers of glycols, di-, oligo- or polyglycols. Preference is given to carrying out the process according to the invention without additional solvents.

The methanol conversion in one reactor pass, which is based by definition on the amount of methanol fed to the reactor and the amount of methanol removed from the reactor, is dependent primarily on whether the process is carried out with an additional solvent or not. When the process is carried out with an additional solvent, it may in some cases constitute the main portion of a liquid reaction mixture, in order to ensure the required concentration of methyl vinyl ether. In this case, the methanol conversion may even be up to 100%.

In the preferred embodiment without use of an additional solvent, the majority of the liquid phase consists of unconverted methanol. This results in the calculation of a low methanol conversion in one reactor pass, which is generally in the range from about 5% to about 25%.

The reaction mixture which is withdrawn from the reactor and comprises methyl vinyl ether, methanol, the basic alkali metal or alkaline earth metal compound, ethyne and in some cases by-products and any solvent added is generally worked up by distillation. In this case, the workup is generally effected by distillation. As a consequence of the relatively low boiling point of methyl vinyl ether of about 6° C., it is typically removed overhead in a column and preferably in a thin-film evaporator, and removed in a downstream column from low boilers such as mainly unconverted ethyne and obtained therefrom as the bottom product. The bottom product of the first column comprises substantially methanol, the basic alkali metal or alkaline earth metal compound and in some cases higher-boiling by-products and also any solvent added, and is preferably recycled to the vinylation reactor. In order to prevent accumulation of by-products and ensure a constant high activity of the basic alkali metal or alkaline earth metal compound, a continuous stream is typically withdrawn as a purge stream and a fresh solution of the basic alkali metal or alkaline earth metal compound is added continuously.

Particular preference is given to a process according to the invention in which
(a) the reaction is carried out in a reactor while feeding the starting compounds, methanol and ethyne,
(b) the reaction mixture obtained by the reaction is decompressed,
(c) the decompressed reaction mixture is separated in a thin-film evaporator into a top stream comprising methyl vinyl ether and a bottom stream comprising methanol and the basic alkali metal or alkaline earth metal compound, and
(d) at least a portion of the bottom stream comprising the alkali metal or alkaline earth metal compound is recycled to the reactor.

The preferred plant for preparing methyl vinyl ether by the process according to the invention comprises a jet loop reactor with external cycle, a postreactor cascade, a thin-film evaporator for removing the methyl vinyl ether formed and a pump for recycling the methanolic solution removed from methyl vinyl ether. In the process according to the invention, methanol, potassium methoxide and ethyne are fed continuously to the jet loop reactor. The feed is via a jet nozzle at the top end which, in conjunction with the concentric insert tube, leads to intensive mixing of the reaction mixture. The reaction mixture to be withdrawn from the external cycle is withdrawn in the lower section of the jet loop reactor, conducted through a cycle pump and a heat exchanger and passed to the jet nozzle. The reaction mixture to be worked up is withdrawn at the top of the jet loop reactor and passed into the postreactor cascade. The reaction mixture leaving the postreactor is decompressed to about atmospheric pressure and cooled, and fed to the thin-film evaporator for separation. A solution comprising the unconverted methanol and potassium methoxide is withdrawn from it via the bottom, and recycled to the external reactor cycle via a recycle pump. In order to prevent accumulation of high boilers, a small stream was discharged as a purge stream. The gas phase withdrawn via the top of the thin-film evaporator is separated in a further column into methyl vinyl ether and an ethynic offgas stream.

The process according to the invention enables the continuous preparation of methyl vinyl ether by vinylating methanol, which is simple to carry out, unproblematic from a safety point of view, especially with regard to the handling of gaseous ethyne, has a high selectivity for methyl vinyl ether and enables an overall high yield and high space-time yield of methyl vinyl ether.

### EXAMPLES

Definitions

The values reported in the examples for ethyne conversion, methanol conversion and space-time yield are defined by the following equations:

$$Ethyne conversion = \frac{{\dot{n}}_{C2H2 , in} - {\dot{n}}_{C2H2 , out}}{{\dot{n}}_{C2H2 , in}}$$

$$Methanol conversion = \frac{{\dot{n}}_{MeOH , in} - {\dot{n}}_{MeOH , out}}{{\dot{n}}_{MeOH , in}}$$

$$Space \text{-} time yield = \frac{{\dot{m}}_{MVE}}{{V}_{reactor}}$$

\dot{m}_{MVE} mass flow rate of methyl vinyl ether produced [g/h]
V_{reactor} volume of the main reactor [l]
\dot{n}_{C2H2,in} molar flow rate of ethyne which is passed into the first reactor, irrespective of its origin, i.e. including recycling [mol/h]
\dot{n}_{C2H2,out} molar flow rate of ethyne which is withdrawn from the last reactor on the outlet side [mol/h]
\dot{n}_{MeOH,in} molar flow rate of methanol which is passed into the first reactor, irrespective of its origin, i.e. including recycling [mol/h]
\dot{n}_{MeOH,out} molar flow rate of methanol, which is withdrawn from the last reactor on the outlet side [mol/h]
   Experimental Plant

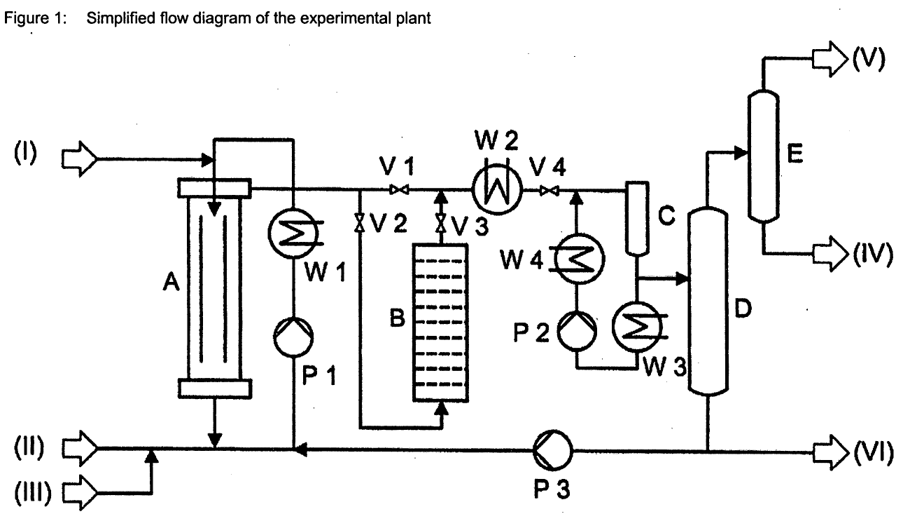

The simplified process flow diagram of the continuous experimental plant is illustrated in FIG. 1. Methanol (II) and a solution of a basic potassium compound (potassium hydroxide in Example 1 and potassium methoxide in Example 2) in methanol (III) are combined with the recycled effluent of reactor A and the recycled stream of the basic potassium compound in methanol, and conducted to the top of the reactor A via the pump P1 and the heat exchanger W1. Ethyne (I) is metered into this feed stream there and passed through a nozzle into the reactor A. The reactor A used was a jacket-heated jet loop reactor having an insert tube and a total volume of 1.6 l. In this reactor, the impulse of the feed stream introduced via the nozzle generates an internal cycle stream. The reactor A is filled during operation to the extent that no continuous gas phase can form. The pump P1 and the heat exchanger W1 already described additionally generate an external cycle stream. The reaction mixture to be worked up is withdrawn at the top of the reactor A. Depending on the execution of the experiment, it was possible to pass the reaction mixture through a postreactor B to increase the ethyne conversion, i.e. to react the remaining dissolved ethyne. The postreactor was a tubular reactor cascade which had a volume of 1.4 l which is provided with plates to prevent gas bubble formation. When the reaction mixture was passed through the postreactor B, the valves V2 and V3 were opened and the valve V1 was closed. When the postreactor B was bypassed, the valves V2 and V3 were closed and the valve V1 was opened. The reaction mixture was cooled using the heat exchanger W2 and decompressed using the decompression valve V4 to about atmospheric pressure. Subsequently, the reaction mixture was cooled further in a quench circuit which comprised a vessel C, a heat exchanger W3, a cycle pump P2 and a further heat exchanger W4. The cooled reaction mixture was withdrawn from the quench circuit and passed into a thin-film evaporator D to remove the unconverted methanol and the basic potassium compound from the methyl vinyl ether and any ethyne present. The solution removed via the bottom, containing the unconverted methanol and the basic potassium compound, was recycled via the pump P3 to the external reactor cycle. In order to prevent accumulation of high boilers, a small stream was discharged as a purge stream (VI). The gas phase withdrawn via the top of the thin-film evaporator D was separated in a further column E into methyl vinyl ether (IV) and an ethynic offgas stream (V).

Experimental Procedure

To start up the experimental plant, the jet loop reactor A was filled with methanol and the basic potassium compound in an appropriate amount, the external cycle was activated and the system was brought to the desired temperature. Subsequently, the postreactor B, when it was used in the experimental plant, the quench circuit, the thin-film evaporator D and the recycling of the methanol and of the basic potassium compound were also activated. By feeding ethyne (I) and methanol, the vinylation reaction was subsequently begun and the desired parameters, for example feed rates, pressure, temperature, cycle and recycle rates, were established in the experimental plant.

The experimental parameters and results of Examples 1 and 2 are reported in Table 1.

The examples show that the process according to the invention, even at a methyl vinyl ether concentration of ≦30% by weight and the reaction in the absence of a continuous gas phase, achieves a high selectivity for methyl vinyl ether and a high yield and high space-time yield for methyl vinyl ether.

TABLE 1
Experimental parameters and results of Examples 1 and 2

| | Example 1 | Example 2 |
|---|---|---|
| Basic potassium compound | potassium hydroxide (KOH) | potassium methoxide (KOMe) |
| | as a 25% by wt. solution in methanol | as a 32% by wt. solution in methanol |
| Feeds | 50 g/h of ethyne | 55 g/h of ethyne |
| | 85 g/h of methanol + 3.66 g/h of KOH | 70 g/h of methanol + 0.14 g/h of KOMe |
| | 500 g/h of recycle stream | 680 g/h of recycle stream |
| Jet loop reactor A: | | |
| Temperature | 110° C. | 110° C. |
| Pressure | 2.0 MPa abs | 2.0 MPa abs |
| Post reactor B: | not in operation | |
| Temperature | | 110° C. |
| Pressure | | 2.0 MPa abs |
| Methyl vinyl ether concentration | 20% by weight | 30% by weight |
| in the liquid phase upstream of the | | |
| decompression valve V4 | | |
| Continuous gas phase | no | no |
| Conversions | 95% ethyne conversion | >99% ethyne conversion |
| | 14 to 16% methanol conversion | 10 to 12% methanol conversion |
| Space-time yield | 70 g/l · h | 80 g/l · h |
| (based on jet loop reactor A) | | |

1. A process for continuously preparing methyl vinyl ether comprising
reacting methanol with ethyne in the liquid phase in the presence of a basic alkali metal compound or alkaline earth metal compound at a temperature of from 40 to 300° C. and at a pressure of from 0.1 to 5 MPa abs,
wherein the reaction is carried out in the absence of a continuous gas phase; and
wherein the reaction is carried out at a methyl vinyl ether concentration in the entire liquid phase of ≦30% by weight.

2. The process of claim 1, wherein the reaction is carried out at a methyl vinyl ether concentration in the entire liquid phase of ≦20% by weight.

3. The process of claim 1, wherein the ethyne is added in an amount of ≦100% of the maximum gas solubility in the liquid phase.

4. The process of claim 1, wherein the reaction is carried out in a jet loop reactor.

5. The process of claim 1, wherein the reaction is carried out in a main reactor while feeding the starting compounds, methanol and ethyne, and one or more postreactors without any further feed of the starting compounds.

6. The process of claim 1, wherein the reaction is carried out at a temperature of from 80 to 150° C.

7. The process of claim 1, wherein the reaction is carried out at a pressure of from 0.5 to 3 MPa abs.

8. The process of claim 1, wherein the basic alkali metal compound or the alkaline earth metal compound is used in the liquid phase in a concentration of from 0.05 to 15% by weight.

9. The process of claim 1, wherein the basic alkali metal compound or the alkaline earth metal compound is potassium methoxide.

10. The process of claim 1, wherein
(a) the reaction is carried out in a reactor while feeding the starting compounds, methanol and ethyne,
(b) the reaction mixture obtained by the reaction is decompressed,
(c) the decompressed reaction mixture is separated in a thin-film evaporator into a top stream comprising methyl vinyl ether and a bottom stream comprising methanol and the basic alkali metal or alkaline earth metal compound, and
(d) at least a portion of the bottom stream comprising the alkali metal or alkaline earth metal compound is recycled to the reactor.

11. The process of claim 2, wherein the ethyne is added in an amount of ≦100% of the maximum gas solubility in the liquid phase.

12. The process of claim 2, wherein the reaction is carried out in a jet loop reactor.

13. The process of claim 3, wherein the reaction is carried out in a jet loop reactor.

14. The process of claim 2, wherein the reaction is carried out in a main reactor while feeding the starting compounds, methanol and ethyne, and one or more postreactors without any further feed of the starting compounds.

15. The process of claim 3, wherein the reaction is carried out in a main reactor while feeding the starting compounds, methanol and ethyne, and one or more postreactors without any further feed of the starting compounds.

16. The process of claim 4, wherein the reaction is carried out in a main reactor while feeding the starting compounds, methanol and ethyne, and one or more postreactors without any further feed of the starting compounds.

17. The process of claim 2, wherein the reaction is carried out at a temperature of from 80 to 150° C.

18. The process of claim 3, wherein the reaction is carried out at a temperature of from 80 to 150° C.

19. The process of claim 4, wherein the reaction is carried out at a temperature of from 80 to 150° C.

20. The process of claim 5, wherein the reaction is carried out at a temperature of from 80 to 150° C.
